(19) United States
(12) Patent Application Publication
(10) Pub. No.: US 2014/0099426 A1
(43) Pub. Date: Apr. 10, 2014

(54) NATURAL COATING FORMULAS AND COMPOSITION FOR COATING TABLETS

(71) Applicant: PHARMAVITE LLC, Northridge, CA (US)

(72) Inventors: Linji Wang, Castaic, CA (US); Louis A. Morin, Tujunga, CA (US); Douglas R. Roper, Redondo Beach, CA (US); Kendall M. Downing, Reseda, CA (US)

(73) Assignee: Pharmavite LLC, Northridge, CA (US)

(21) Appl. No.: 13/648,891

(22) Filed: Oct. 10, 2012

(51) Int. Cl. A61K 47/02; A61K 9/70

(52) U.S. Cl. 427/2.23; 514/769

(57) ABSTRACT

A solid dosage form coating composition including gray oyster shell powder in a form suitable to be coated on a solid dosage form. A method including coating a solid dosage form with a coating composition comprising gray oyster shell powder; and drying the coating composition into a film.

### FIELD

Solid dosage form coatings.

### BACKGROUND

Film coatings for solid dosage forms such as tablets in the dietary, nutritional and pharmaceutical markets are a common practice in those various markets. Purposes for coating a tablet or caplet with a film is to protect the active ingredient(s), eliminate dust for packaging, improve appearance, improve swallowability, extend the shelf life and reduce objectionable odor and spots. A coating formulation typically contains a viscous polymer for forming a film, an opacifier to inhibit light from penetrating the coating film, a plasticizer to improve sprayability of a polymer during a coating process and a color agent and a filler or stabilizer to enhance a stability of a coating solution during a coating process.

Polymers include natural and synthetic polymers. Examples of natural polymers include starch, seaweed extract such as carigeenan, gums for plants and fungi. Examples of semi-natural polymers are chemically modified starch, hydroxypropyl methylcellulose (HPMC), hydroxyethyl cellulose and hydroxyethylmethyl cellulose. Examples of synthetic polymers include polyvinyl alcohol and polyvinyl alcohol esters.

Examples of opacifiers include titanium dioxide, zinc oxide, ferric oxide and calcium carbonate.

Examples of plasticizers include polyethylene glycol, polysorbate, glycerin, medium chain triglycerides, food oils, other lipids with low melting points, and triethyl citrate. Examples of colorants include natural and artificial colors. Examples of stabilizers and bulk agents include talc and maltodextrin.

Solid dosage forms such as tablets are typically coated in a pan in a controlled environment, where a temperature, airflow, pan rotation, tablet bed thickness, and coating solution spray rate are all measured. The coating powder that represents the coating formulas is generally mixed into water and then sprayed onto the tablets in a form of atomized droplets. The tablets are tumbled inside the pan in the presence of hot and passing air. As the tablets are tumbled in the presence of the hot and passing air, the coating droplets on the tablet dries and a film is formed on a surface of the tablet.

Many tablets containing nutritional supplement or pharmaceutical active cores can contain discolorations and/or dark color spots either initially or at a later stage of a shelf life. The appearance of discolorations and/or dark spots affects tablet quality and consumer acceptance. To minimize the visibility of discolorations and/or dark spots, tablet coating compositions or formulations often contain titanium dioxide. The heat effects of titanium dioxide, however, have recently been questioned.

### DETAILED DESCRIPTION

A solid dosage form coating composition, a method of use of a solid dosage form coating, and a method of forming a coating composition on a solid dosage form are described. As used herein, a solid dosage form is a tablet, caplet or softgel capsules for oral, including ingestible, buccal and sublingual, administration to a mammal including a human.

In one embodiment, the solid dosage form coating composition includes gray oyster shell powder in a form suitable to be coated on a solid dosage form. Gray oyster shell is a natural or non-manufactured product that had been previously used as a source of calcium in, for example, a core of a calcium tablet. Gray oyster shell, in one aspect, acts as a light inhibiting or blocking agent and imparts the darker shade to the coating which not only inhibits light but also masked dark spots in the core solid dosage form (e.g., core tablet). Calcium which is present in both gray and white oyster shell powder is a heavy metal element and tends to block light rays. The natural gray color of the gray oyster shell powder exhibits surprisingly better effect in blocking light than white shell oyster powder. The improved effect is believed to be derived from the gray color of the oyster shell powder which blocks more light rays than white oyster shell; the gray color masks dark spots and discoloration of the core tablets; and the coated tablets have a more natural food look. Practical achievement in tablet appearance is one benefit. Gray oyster shell powder also provides for acceptable masking of dark spots and discolorations to eliminate titanium dioxide from a coating composition.

In one embodiment, a representative formulation of a solid dosage form coating composition including gray oyster shell powder, also includes a cellulose derivative or a cellulose gel. One example of a cellulose gel is hydroxypropylmethyl cellulose (HPMC) Very Low Viscosity (VLV) hypromellose™, commercially available from the Dow Chemical Company, of Midland, Mich. HPMC VLV is hydroxypropyl methyl cellulose CAS No. 9004-65-3 with 27 percent to 30 percent methoxyl substituents and 4 percent to 7.5 percent hydroxypropyl substituents. A compositional breakdown of HPMC VLV is 85-99 percent hydroxpropyl methyl cellulose; 0.5-5 percent sodium chloride (CAS No. 7647-14-5); and 1-10 percent water (CAS No. 7732-18-5). In another embodiment, a suitable cellulose gel is another form of hydroxypropyl methylcellulose (HPMC), including but not limited to METHOCEL™ HPMC, commercially available from the Dow Chemical Company (e.g., with 24 percent methoxyl substituents and 9 percent hydroxypropyl substituents, “HPMC 24:9”) or BENECEL™ HPMC, commercially available from Ashland Aqualon Functional Ingredients of Wilmington, Del. In a further embodiment, a suitable cellulose gel is a combination of hydroxypropyl methyl cellulose grades including HPMC VLV and one or more other grades of HPMC (e.g., a 80:20, 60:40, 50:50, 40:60 mixtures of HPMC VLV:HPMC), or HPMC (e.g., HPMC VLV) combined with one or more other polymers including, but not limited to, hydroxyethyl cellulose, ethyl cellulose, hydroxypropyl cellulose, povidone, sodium carboxy methyl cellulose, a hydrolyzed guar gum, an acacia bean gum or a seaweed gum.

In addition to the gray oyster shell powder and cellulose gel, in one embodiment, a solid dosage form coating composition includes other natural or non-manufactured components including natural colors, such as coffee, cocoa powder, jalapeno, tea leaves, spirulina algae, berry extract, cabbage, beet or fruit extract. Natural or non-manufactured sweeteners may also be included. An example of a natural or non-manufactured sweetener includes, but is not limited to, stevia extract. A natural flavor that, in one embodiment, is also included in a coating composition includes, but is not limited to, an extract of mint, orange or vanilla bean. The addition of sweeteners and/or flavors to a coating composition improves the taste of a solid dosage form while it is inside the mouth.

A representative formulation of a solid dosage form coating composition is as follows:

| | |
|---|---|
| Cellulose gel or combination | 25-90% by weight; |
| Fractionated Coconut oil | 2-20% by weight; |
| Gray oyster shell powder | 5-40% by weight; |
| Natural colorants or color blend | 0.1-15% by weight; |
| Natural sweetener and flavors | 0.1-15% by weight. |

In another embodiment, a composition includes:

| | |
|---|---|
| HPMC VLV | 64% by weight; |
| Fractionated Coconut oil | 7% by weight; |
| Gray oyster shell powder | 20% by weight; |
| Natural colorants or color blend | 8% by weight; |
| Natural sweetener and flavors | 1% by weight. |

The above-described composition provides a natural product coating composition for a solid dosage form. In one embodiment, all the ingredients may be selected such that they are considered natural products. Such ingredients include a cellulose gel, a gray oyster shell powder, natural or non-manufactured colorants, flavors and sweeteners. A natural coating composition such as described appeals to the dietary, nutritional and pharmaceutical industry.

In one embodiment, the ingredients of a solid dosage form coating composition are combined in dry form. The dry formulation is weighed and mixed with purified water in a stainless steel tank to form a solution. A solution typically contains between 5 and 30 percent by weight solids and 10 to 95 percent water. Next, the solution is pressurized and sprayed in a form of atomized droplets onto tumbling core tablets in a pan. This is accomplished in controlled heated and dry conditions. Common coating conditions are coating pan rotation speed: 2 to 15 round per minutes, tablet temperature at 20° C. to 65° C., airflow: 2000 to 6000 cubic feet per minute (cfm). The temperature, dryness and airflow are maintained such that once a coating solution droplet touches down on the surface of a tablet, it is dried and forms a film on the surface and any water evaporates. In a period of 20 to 200 minutes of tablets tumbling inside the coating pan, many coating solution droplets touch down onto each tablet and thus form a film including a coating over each tablet. When a coating process is completed, each tablet is coated with the coating composition.

The above-described coating process may be done as a batch process or a continuous process. In a batch process, a coating pan is loaded with a desired amount of core tablets; the tablets are coated with desired amount of a coating composition and then discharged when coated as finished product. In a continuous process, core tablets are continuously loaded into a coating pan and tumbling tablets continuously coated and then unloaded tablets from the pan.

Embodiments of coating compositions are described in the following examples:

### EXAMPLE 1

A formula to coat tablets with blue film.

| | |
|---|---|
| HPMC VLV | 71.5% |
| Fractionated coconut oil | 7.2% |
| Gray oyster shell powder | 20% |
| Billberry extract | 0.30% |
| Stevia extract | 1% |

### EXAMPLE 2

A formula to coat tablets with green film.

| | |
|---|---|
| HPMC VLV | 31.95% |
| HPMC 24:9 | 31.95% |
| Fractionated coconut oil | 7.1% |
| Gray oyster shell powder | 20.0% |
| Spirulina powder | 8% |
| Stevia extract | 1% |

### EXAMPLE 3

A formula to coat tablets with purplish red film.

| | |
|---|---|
| HPMC VLV | 31.95% |
| HPMC 24:9 | 31.95% |
| Fractionated coconut oil | 7.1% |
| Gray oyster shell powder | 20.0% |
| Red beet extract | 8% |
| Stevia extract | 1% |

### EXAMPLE 4

A formula to coat tablets with beige colored film.

| | |
|---|---|
| HPMC VLV | 31.95% |
| HPMC 24:9 | 31.95% |
| Fractionated coconut oil | 7.1% |
| Gray oyster shell powder | 20.0% |
| Coffee powder | 8% |
| Stevia extract | 1% |

In another embodiment, a method of use is described. Representatively, a method of use of a solid dosage form such as a tablet including a coating composition includes placing the tablet in a mouth of a mammal (e.g., human) and swallowing the tablet with the aid of a drink. In another embodiment, a tablet may be intended for buccal or sublingual administration. In such case, rather than swallowing the tablet, the tablet will remain in the mouth of the mammal until it disintegrates or dissolves.

In the description above, for the purposes of explanation, numerous specific details have been set forth in order to provide a thorough understanding of the embodiments. It will be apparent however, to one skilled in the art, that one or more other embodiments may be practiced without some of these specific details. The particular embodiments described are not provided to limit the invention but to illustrate it. The scope of the invention is not to be determined by the specific examples provided above but only by the claims below. In other instances, well-known structures, devices, and operations have been shown in block diagram form or without detail in order to avoid obscuring the understanding of the description. Where considered appropriate, reference numerals or terminal portions of reference numerals have been repeated among the figures to indicate corresponding or analogous elements, which may optionally have similar characteristics.

It should also be appreciated that reference throughout this specification to “one embodiment”, “an embodiment”, “one or more embodiments”, or “different embodiments”, for example, means that a particular feature may be included in the practice of the invention. Similarly, it should be appreciated that in the description various features are sometimes grouped together in a single embodiment, figure, or description thereof for the purpose of streamlining the disclosure and aiding in the understanding of various inventive aspects. This method of disclosure, however, is not to be interpreted as reflecting an intention that the invention requires more features than are expressly recited in each claim. Rather, as the following claims reflect, inventive aspects may lie in less than all features of a single disclosed embodiment. Thus, the claims following the Detailed Description are hereby expressly incorporated into this Detailed Description, with each claim standing on its own as a separate embodiment of the invention.

What is claimed is:

1. A solid dosage form coating composition comprising gray oyster shell powder in a form suitable to be coated on a solid dosage form.

2. The solid dosage form coating composition of claim 1, further comprising a cellulose gel.

3. The solid dosage form coating composition of claim 2, wherein the cellulose gel comprises hydroxypropyl methyl cellulose.

4. The solid dosage form coating composition of claim 2, wherein the cellulose gel comprises hydroxypropyl methyl cellulose very low viscosity.

5. The solid dosage form coating composition of claim 4, wherein the cellulose gel further comprises another grade of HPMC.

6. The solid dosage form coating composition of claim 2, further comprising a non-manufactured colorant.

7. The solid dosage form coating composition of claim 6, wherein the non-manufactured colorant comprises coffee.

8. The solid dosage form coating composition of claim 2, further comprising a non-manufactured sweetener.

9. The solid dosage form coating composition of claim 8, wherein the sweetener comprises stevia extract.

10. The solid dosage form coating composition of claim 1, further comprising:
hydroxypropyl methyl cellulose;
fractionated coconut oil; and
a non-manufactured colorant.

11. The solid dosage form coating composition of claim 10, further comprising a non-manufactured sweetener or flavor.

12. The solid dosage form coating composition of claim 11, wherein the sweetener comprises stevia extract.

13. The solid dosage form coating composition of claim 10, wherein the hydroxypropyl methyl cellulose comprises hydroxypropyl methyl cellulose very low viscosity.

14. A method comprising:
coating a solid dosage form with a coating composition comprising gray oyster shell powder; and
drying the coating composition into a film.

15. The method of claim 14, wherein the coating composition comprises a cellulose gel.

16. The method of claim 15, wherein the cellulose gel comprises hydroxypropyl methyl cellulose.

17. The method of claim 15, wherein the cellulose gel comprises hydroxypropyl methylcellulose VLV.

18. The method of claim 15, wherein the cellulose gel comprises a combination of hydroxypropyl methyl cellulose VLV and another grade of hydroxypropyl methyl cellulose.

19. The method of claim 14, wherein the coating solution comprises 70 to 95 percent water.

20. The method of claim 14, wherein the coating composition further comprises a non manufactured colorant.

21. The method of claim 14, wherein the coating composition further comprises a non-manufactured sweetener.

22. The method of claim 14, wherein the coating composition comprises a solution comprising water, hydroxypropyl methyl cellulose, fractionated coconut oil, a non-manufactured colorant and a natural sweetener, stevia extract.
